(19) United States
(12) Patent Application Publication
(10) Pub. No.: US 2026/0074031 A1
(43) Pub. Date: Mar. 12, 2026

(54) PRINCIPAL INVESTIGATOR IDENTIFICATION FOR CLINICAL TRIAL

(71) Applicant: Techpeace Technologies, Palo Alto, CA (US)

(72) Inventors: Jason Carlin, Chester, NV (US); Paulomi Patel, Livingston, NJ (US)

(73) Assignee: Techpeace Technologies, Palo Alto, CA (US)

(21) Appl. No.: 19/326,489

(22) Filed: Sep. 11, 2025

Related U.S. Application Data
(60) Provisional application No. 63/693,855, filed on Sep. 12, 2024.
(60) Provisional application No. 63/693,868, filed on Sep. 12, 2024.

(51) Int. Cl. G16H 10/20; G16H 10/60

(52) U.S. Cl. CPC G16H 10/20; G16H 10/60

(57) ABSTRACT

A method of identifying a principal investigator for a clinical trial includes identifying candidate principal investigators based on similarity of past clinical trials of each of the candidate principal investigators to the clinical trial. The method also includes accessing open-source information to identify patients associated with each of the candidate principal investigators, and recording demographic information for the patients associated with each of the candidate principal investigators. A principal investigator is identified for the clinical trial based on a match between the demographic information for the patients associated with each of the candidate principal investigators and demographic requirements defined for the clinical trial.

### RELATED APPLICATIONS

This application claims the benefit under 35 U.S.C. § 119(e) of U.S. Provisional Application No. 63/693,868 entitled “PRINCIPAL INVESTIGATOR IDENTIFICATION FOR CLINICAL TRIAL,” filed Sep. 12, 2024. This application claims the benefit under 35 U.S.C. § 119(e) of U.S. Provisional Application No. 63/693,855 entitled “PRINCIPAL INVESTIGATOR IDENTIFICATION FOR CLINICAL TRIAL,” filed Sep. 12, 2024, the entire contents of each are incorporated herein by reference by their entirety.

### FIELD OF THE DISCLOSURE

The present disclosure relates generally to the management of a clinical trial. More specifically, the present disclosure relates to identification of a principal investigator for a clinical trial.

### BACKGROUND OF THE DISCLOSURE

Generally, before a new or modified drug, biological product, or medical device—generally “medical product”—is made available to the public, it must obtain approval from the Food and Drug Administration (FDA). The process of obtaining FDA approval can involve a clinical trial of the new or modified medical product to assess its efficacy and safety. A clinical trial is a research study conducted according to a protocol that specifies eligibility criteria for patients who voluntarily participate in the trial, dosages, length of the study, and other parameters. There are different phases of clinical trials (e.g., Phase 1, Phase 2, Phase 3) depending on the stage of development of the medical product. A clinical trial typically involves one or more principal investigators (e.g., physician, dentist) enrolling patients in the trial and following the established protocols to test the medical product within the enrolled group. While the clinical trial is designed to assess the success of the medical product, the success of the clinical trial can hinge on the enrollment of a sufficient number of qualified patients in the trial.

### SUMMARY

According to an exemplary embodiment, a method of identifying a principal investigator for a clinical trial includes identifying candidate principal investigators based on similarity of past clinical trials of each of the candidate principal investigators to the clinical trial. The method also includes accessing open-source information to identify patients associated with each of the candidate principal investigators, and recording demographic information for the patients associated with each of the candidate principal investigators. A principal investigator is identified for the clinical trial based on a match between the demographic information for the patients associated with each of the candidate principal investigators and demographic requirements defined for the clinical trial.

There has thus been outlined, rather broadly, the features of the disclosed subject matter in order that the detailed description thereof that follows may be better understood, and in order that the present contribution to the art may be better appreciated. There are, of course, additional features of the disclosed subject matter that will be described hereinafter and which will form the subject matter of the claims appended hereto. It is to be understood that the phraseology and terminology employed herein are for the purpose of description and should not be regarded as limiting.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. In the figures:

FIG. 1 is a process flow of a method of selecting a principal investigator for a clinical trial according to one or more embodiments;

FIG. 2 shows aspects of the method of FIG. 1 related to determining the past enrollment success score according to one or more embodiments;

FIG. 3 shows aspects of the method of FIG. 1 related to determining the eligible patient score according to one or more embodiments;

FIG. 4 shows additional aspects of the method of FIG. 1 related to determining the eligible patient score according to one or more embodiments;

FIG. 5 shows additional aspects related to referral sources for eligible patients according to one or more embodiments;

FIG. 6 is a process flow of a method of recording demographic information for patients of candidate principal investigators according to one or more embodiments

FIG. 7 shows aspects of the method of FIG. 1 related to determining the distraction score according to one or more embodiments; and

FIG. 8 is a block diagram of processing circuitry used to implement the method of FIG. 1 according to one or more embodiments.

### DETAILED DESCRIPTION OF THE INVENTION

Clinical trials can be an essential aspect of obtaining regulatory approval for a medical product (e.g., drug, biological product, or medical device). A clinical trial requires at least one principal investigator whose responsibility it is to enroll eligible patients and manage the clinical trial. Both eligibility of the participants (e.g., age range, gender, medical history, ineffectiveness of previous medication) and processes involved in the trial management are set out by the protocol approved for the clinical trial. The protocol also includes a duration for the clinical trial. Historically, a large percentage of clinical trials are delayed or closed due to problems with recruitment of eligible patients by the principal investigator(s). Thus, for a pharmaceutical company or other enterprise seeking FDA approval of its medical product, successful completion of the clinical trial can be heavily dependent on identifying the principal investigator(s) who will quickly enroll eligible patients and see the clinical trial through to completion.

Provided herein are techniques to identify one or more principal investigators for a clinical trial. Aspects of the techniques relate to obtaining a score for each potential principal investigator based on previous success with clinical trial enrollment, eligible patient availability, and lack of involvement in other clinical trials that may divert eligible patients. Previous success can refer to eligible patient enrollment success of the potential principal investigator in past, similar clinical trials. Eligible patient availability can refer to the number of patients eligible or partially eligible for the clinical trial who are patients of the potential principal investigator, of the medical facility in which the potential principal investigator practices, or within an area (e.g., same first three digits of the zip code) of the medical facility. Lack of other clinical trials can refer to the fact that even a potential principal investigator with a large number of available eligible patients and previous success may be given a score reduction if other contemporaneous clinical trials are ongoing that may siphon eligible patients. Such factors may be used to obtain an overall score for each potential principal investigator that is then used to rank the potential principal investigators. The processes involved in obtaining the score for each potential principal investigator may rely on one or more machine learning algorithms.

The inventors have recognized and appreciated the need for a data-driven approach to identifying principal investigators for clinical trials. The inventors also appreciated that, while publicly available internet-based data and insurance data may provide some indicators of potential success of a principal investigator, there are challenges to gleaning useful information based on the distributed and uncoordinated presentation of the data. In particular, for example, certain data that can be used to analyze principal investigators may be incomplete and/or missing. As a non-limiting example, a website listing current and historical clinical trials may not always list the principal investigators and clinical trial sites. This listing may be combined with another website indicating payments provided for current and historical clinical trials in order to identify the principal investigator and site associated with clinical trials of interest. The identification information may be used with the payment and/or other information to ascertain past success of candidate principal investigators, for example. As another example, data may need to be processed in unconventional ways in order to generate data that can be used to analyze principal investigators. By using various machine learning techniques in combination, the technical challenge of coordinating data from different internet sources to ultimately provide a standardized score for each potential principal investigator is made practicable. Accordingly, the techniques provide improvements to computerized technology for analyzing and planning clinical trials.

In the following description, numerous specific details are set forth regarding the systems and methods of the disclosed subject matter and the environment in which such systems and methods may operate, etc., in order to provide a thorough understanding of the disclosed subject matter. In addition, it will be understood that the examples provided below are exemplary, and that it is contemplated that there are other systems and methods that are within the scope of the disclosed subject matter.

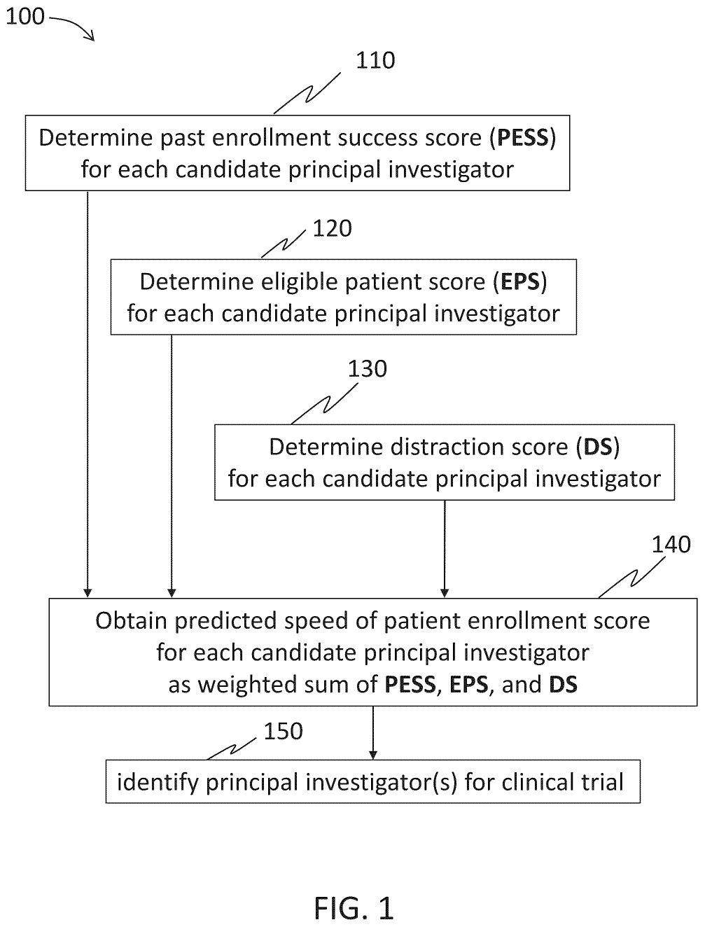

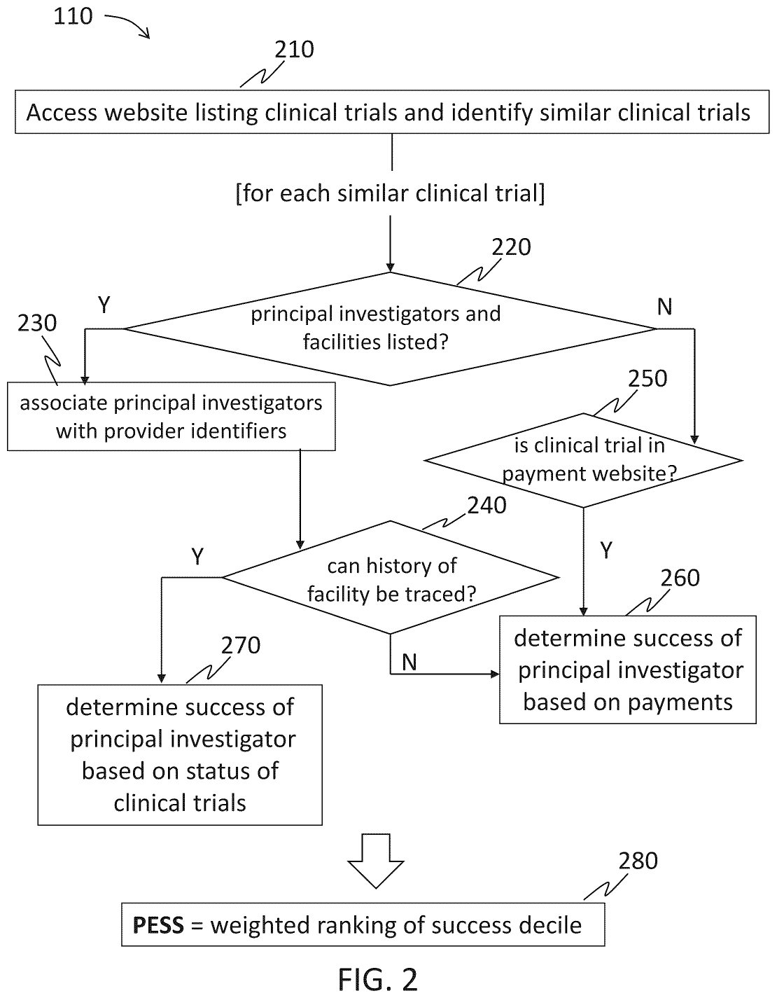

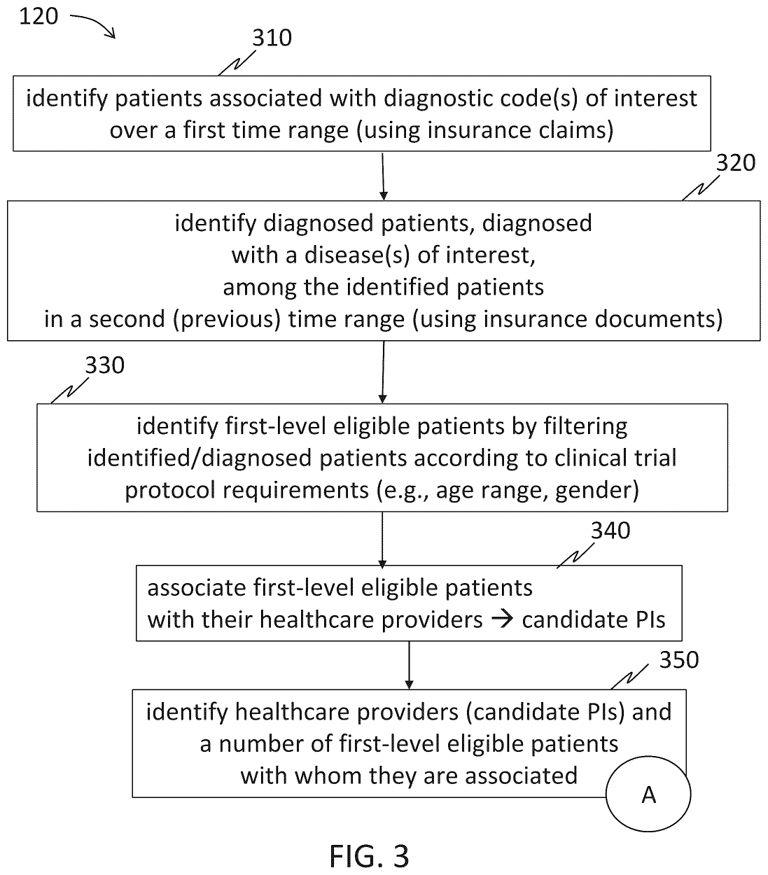

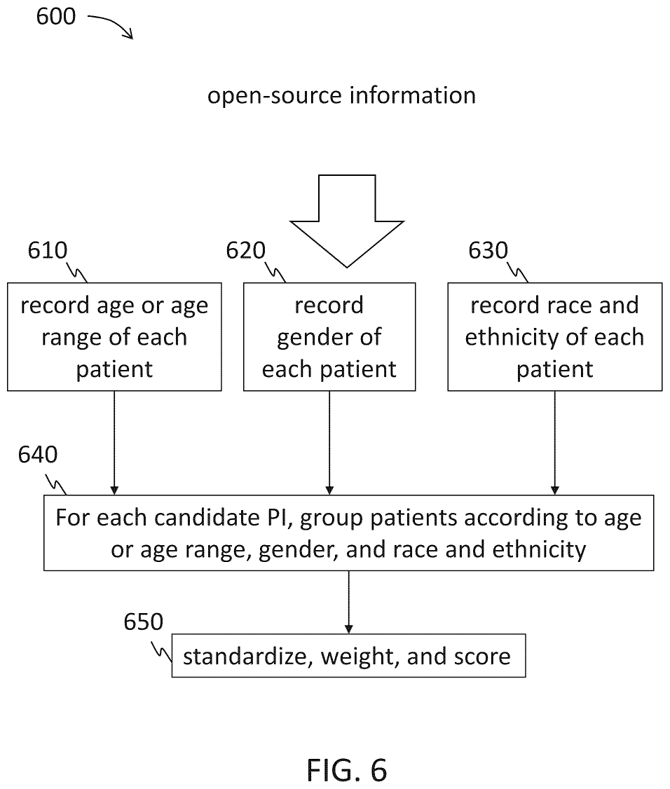

FIG. 1 is a process flow of a method 100 of selecting a principal investigator for a clinical trial according to one or more embodiments. At 110, determining a past enrollment success score (PESS) for each candidate principal investigator (PI) includes processes detailed with reference to FIG. 2. This score considers whether a candidate PI successfully recruited patients for past clinical trials. At 120, determining an eligible patient score (EPS) for each candidate PI includes processes detailed with reference to FIGS. 3-5. This score considers the patients who are eligible for the clinical study who are patients of the candidate PI or are available to join a clinical trial run by the candidate PI due to their connection with the same healthcare facility or area as the candidate PI. At 130, determining a distraction score (DS) for each candidate PI includes processes detailed with reference to FIG. 6. This score considers whether each candidate PI has other clinical trials that may divert enrollment of available eligible patients away from the clinical trial of interest, which may be referred to as the target clinical trial. At 140, obtaining a predicted speed of patient enrollment score for each candidate PI may be based on a weighted sum of the PESS, EPS, and DS. The predicted speed of patient enrollment score for each candidate PI may then be used, at 150, to identify one or more PIs for the target clinical trial. It should be appreciated that the acronyms used herein, such as PESS, EPS and DSS, are used for explanatory purposes only and are not intended to limit the scope of the techniques described herein.

FIG. 2 details aspects of the method 100 related to determining the PESS at 110 according to one or more embodiments. At 210, accessing one or more websites that list clinical trials and identifying similar clinical trials to the target clinical trial may involve use of a database of clinical trials, such as that provided via a website like clinicaltrials.gov, for example. Identifying similar clinical trials may include web scraping to obtain data from the website(s) and filtering to isolate data related to parameters specified to identify similar clinical trials (e.g., diagnosis, name of condition, class of medication being tested, age range of eligible participants). Generative Artificial Intelligence (GenAI) may be used to generate a list of similar trials to the target clinical trial based on parameters describing the target clinical trial.

Processes at 220-270 may be performed for each similar clinical trial identified at 210. At 220, a check is done of whether one or more Pls and facilities are listed in association with the similar clinical trial. If so, at 230, the PIs may be associated with respective provider identifiers such as a National Provider Identifier (NPI) in the NPI database, for example. The association at 230 may result from implementing fuzzy matching on the name of the similar clinical trial and demographic information (e.g., city, state, zip code, geographic code, specialty) included for the similar clinical trial. At 240, a check is done of whether a history of the similar clinical trial at each of the facilities can be traced. If the check at 240 indicates that information for the similar clinical trial cannot be traced, processes at 260 are performed. As indicated in FIG. 2, the processes at 260 may be reached another way, as well.

If the check at 220 indicates that the similar clinical trial listed at the website (accessed at 210) does not list PIs and/or facilities, then the principal investigators need to be identified by the system using other data. In some embodiments, a check may be done, at 250, of whether the similar clinical trial is listed in another database, such as in a payment website. The payment website may be openpaymentsdata.cms.gov or a similar website that indicates payments made by pharmaceutical or medical device enterprises to facilities and providers for clinical trials. The processes at 250 may include implementing fuzzy matching of the name of the similar clinical trial with payments listed in the payment website and/or implementing web scraping to identify an official title that may provide a better match to the name of the similar clinical trial. If the similar clinical trial is found in the payment website (according to the check at 250), the PIs may be identified using this additional data, and the processes at 260 may be performed.

At 260, determining success of the PIs associated with the similar clinical trial is based on all payments associated with the similar clinical trial. The payment website may be used to determine an average payment amount to PIs for the clinical trial. Any of the PIs identified at 220 or 250 whose payment exceeds that average are deemed successful for that clinical trial. Any of the PIs whose payments are less than the average are deemed unsuccessful.

If the check at 240 indicates that similar clinical trial history can be traced for one or more facilities associated with the similar clinical trial, processes at 270 may be performed. At 270, each facility's success, which is used as an indication of the associated PI's success, is determined based on the last status indicated for the similar clinical trial at the facility. For example, facility history indicating “active, not recruiting” or “completed” may be deemed to indicate a successful PI, while facility history indicating “terminated,” “withdrawn,” or “suspended” may be deemed to indicate an unsuccessful PI. Facility history indicating “recruiting” is not used as an indication of either success or lack of success. The facility history may be triangulated from the website that lists clinical trials (e.g., clinicaltrials.gov) and the payment website (e.g., openpaymentsdata.cms.gov).

When the processes at 220-270 are completed for each of the similar clinical trials, PESS may be determined at 280. Specifically, a decile ranking of PIs may be obtained based on the number of successful similar clinical trials of each PI. A weighting (e.g., 50 percent) may then be applied to the decile score of each PI to determine the PESS of that PI.

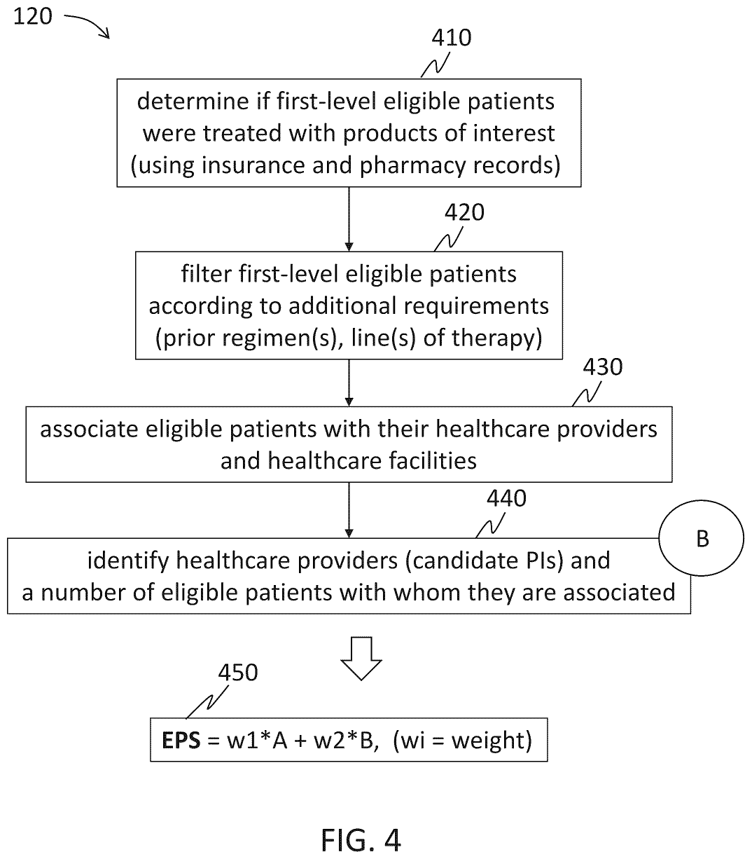

FIGS. 3 and 4 detail aspects of the method 100 related to determining the EPS at 120 according to one or more embodiments. FIG. 3 pertains to diagnosed patients associated with candidate PIs. At 310, identifying patients associated with one or more diagnostic codes of interest over a first time range may involve searching insurance claims. The diagnostic codes of interest may be defined by the protocol of the target clinical trial, for example. At 320, earlier insurance claims may be accessed for the identified patients (identified at 310), to identify diagnosed patients, diagnosed with disease(s) of interest among the identified patients, during an earlier period. That is, earlier insurance claims may be used at 320. The earlier claims may be from a second time range preceding the first time range.

At 330, identifying first-level eligible patients includes filtering identified and diagnosed patients (from 310 and 320) according to protocol requirements (e.g., age range, gender) of the target clinical trial. At 340, associating the first-level eligible patients with their healthcare providers indicates candidate PIs with patients who may be eligible for the target clinical trial. At 350, identifying the candidate PIs and the number of first-level eligible patients with whom they are associated (at 340) provides a result indicated as A.

FIG. 4 pertains to treated patients associated with candidate PIs. At 410, determining if first-level eligible patients (from the processes of FIG. 3) were treated with products of interest may involve searching insurance and pharmacy records. Products of interest may be defined by the protocol of the target clinical trial. For example, the target clinical trial may specify that eligible patients are ones who have tried a prior drug.

At 420, the processes include filtering the first-level eligible patients according to additional requirements of the target clinical trial. These additional requirements may include prior regimen(s) or line(s) of therapy. The result of the filtering may be patients deemed to be eligible patients for the target clinical trial. At 430, associating eligible patients and their healthcare providers (i.e., candidate PIs from 340) with healthcare facilities indicates candidate PIs and potential facilities for the target clinical trial. At 440, identifying the candidate PIs and the number of eligible patients with whom they are associated (at 430) provides a result indicated as B.

At 450, the weighted sum of the results A and B may be the EPS for each candidate PI. Specifically, EPS may be obtained as:

$$EPS = w 1 * A + w 2 * B \qquad [ EQ . 1 ]$$

Each wi (with i=1 or 2) is the weight associated with the respective result A or B. The weights w1 and w2 may be expressed as a percentage (e.g., w1=w2=50 percent). In some embodiments, the weights wi may be adjusted based on one or more factors. For example, a machine learning algorithm may be trained according to prior recruitment success and may determine the weights wi. The weights wi may alternately or additionally modified based on the protocol requirements and how difficult they are to satisfy. For example, if eligible patients for a given study are required to have completed treatment with a product that was only administered to a small percentage of the patient population, w2 may have a higher value than w1. On the other hand, if the protocol of the target clinical trial requires patients to have tried a common product or one that can be administered to ready the patient for the target trial, then w1 may have a higher value than w2.

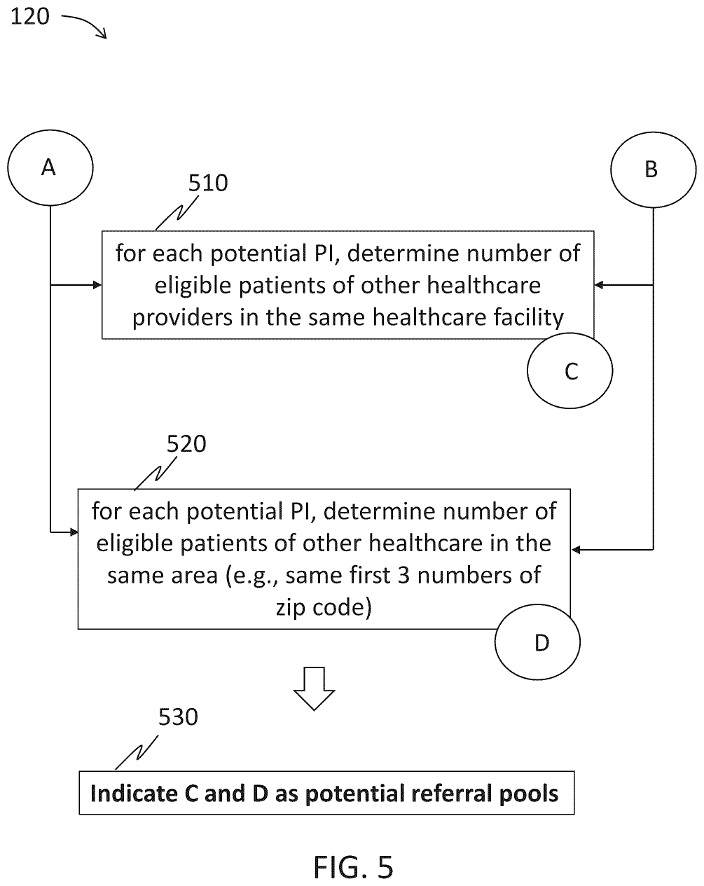

FIG. 5 pertains to referral possibilities and determination of EPS. While the results (C and D) of the processes shown in FIG. 5 may not be used in the EPS score, they may be helpful in considering which candidate PIs have a larger pool of eligible patients to draw from. At 510, for each candidate PI (identified at 340 and 430), the processes include determining a number of eligible patients of other healthcare providers at the same facility. The processes at 510 may include finding eligible patients who are treated at facilities with the same 5-digit zip code as that of each candidate PI's facility but are not associated with the candidate PI (at 350 and 440). For each candidate PI, the number of these eligible patients who are not patients of the candidate PI but are treated in the same zip code may be indicated as result C.

At 520, for each candidate PI (identified at 340 and 430), the processes include determining a number of eligible patients of other healthcare providers at nearby facilities. The processes at 520 may include finding eligible patients who are treated at facilities with at least the same first three digits of the zip code (but not all five digits) as that of each candidate PI's facility. For each candidate PI, the number of these eligible patients who are not patients of the candidate PI but are treated in the same area may be indicated as result D. At 530, indicating C and D as potential referral pools from which the associated candidate PI may recruit patients for the target trial may provide additional insight into eligible patients and potential recruitment success of the candidate PIs.

An additional factor that may be considered for the eligible patients indicated in results C and D as potential referral patients for the target clinical trial is experience of their healthcare provider with any current or past clinical trials. Because a healthcare provider with clinical trial experience may be more likely to refer a patient for participation in a clinical trial, eligible patients who are not patients of the candidate PI but are treated in the same zip code (those in result C) and eligible patients who are not patients of the candidate PI but are treated in the same area (those in result D) may be considered to be more likely candidates for participation in the target clinical trial based on the clinical trial experience of their healthcare providers. Thus, if C and D are considered in the EPS, weightings may be adjusted according to healthcare provider clinical trial experience.

FIG. 6 is a process flow of a method 600 of recording demographic information for patients of candidate PIs according to one or more embodiments. Some clinical trials may require a particular mix of demographic parameters for the patients who participate in the clinical trial. For example, a manufacturer may want to ensure that clinical trials were performed on women, as well as men, for a new blood pressure medication. As another example, the clinical trial may be needed for approval of a new drug or device by the Food and Drug Administration. In this case, the clinical trial may include and may need to comply with a diversity action plan. In these cases, determining an eligible patient score (EPS) for candidate PIs without considering the demographics of patients associated with those PIs may be unhelpful in ultimately selecting a PI who can successfully enroll not only the number but also the type of patients needed for the target clinical trial. While all patients, rather than only eligible patients, may be considered, the method 600 informs the familiarity and access of a candidate PI relative to a demographic of interest and suggests the case with which eligible patients of the demographic may be on-boarded in the target trial by the candidate PI.

For each candidate PI, open-source information may be accessed (e.g., via web scraping) to identify patients and categorize those patients. Exemplary sources of demographic information include the Centers for Medicare and Medicaid Services (CMS) databases that provide claims data and research payment data, National Plan and Provider Enumeration System (NPPES), and data on current and past clinical trials. As noted with reference to FIG. 5, along with a given candidate PI's own patients and their demographic information, demographic information for patients of other healthcare providers who may be referral sources may be considered. Thus, demographic information for patients treated at facilities with at least the same first three digits of the zip code associated with a candidate PI's address may be considered. In addition, population diversity (e.g., in the zip code of a candidate PI's address) according to the latest census data may be used as a gauge how closely the diversity of a candidate PI's patients reflects the diversity in the candidate PI's area.

The processes may include recording age or age range of each patient, at 610, gender of each patient (at 620), and race and ethnicity of each patient (at 630). At 640, patients of each candidate PI (and, optionally, patients in a potential pool) may be grouped according to age or age range, gender, and race and ethnicity. That is, each candidate PI may be associated with demographic statistics for their patients. Thus, for example, the percentage of patients associated with a candidate PI that are female and between ages 65 and 74 can be determined from the recorded information (at 610, 620, 630).

At 650, the processes may begin with determining a percentage of coverage of required demographic criteria for a target clinical trial (based on the information at 610, 620, 630). For example, if a candidate PI has 100 patients, half of whom are women and half of whom are men, that may represent 100 percent coverage of gender criteria for the target clinical trial. The processes at 650 may include standardizing the coverage values based on census data (e.g., for the area with the same first three digits of the zip code associated with a candidate PI's address). Thus, for example, if a candidate PI has 80 percent coverage of race criteria for a target clinical trial, the fact that the population in the area has 30 percent of the racial diversity required, the candidate PI's race coverage may be adjusted up, since the candidate PI's patients evidence more racial diversity than the area. Different weights may be assigned to candidate PI-specific coverage, coverage of patients in the area (e.g., from the CMS database), and coverage of the general population in the area.

According to some embodiments, the demographic information may be used as an additional factor (in addition to the weighted sum determined as part of the processes 140 of the method 100 (FIG. 1)) to select a PI from among the candidate PIs. According to some embodiments, the demographic information may be used to weight the scores (at 140) to identify the PI (at 150) who is most likely to enroll the required number and demographics of eligible patients in the target clinical trial.

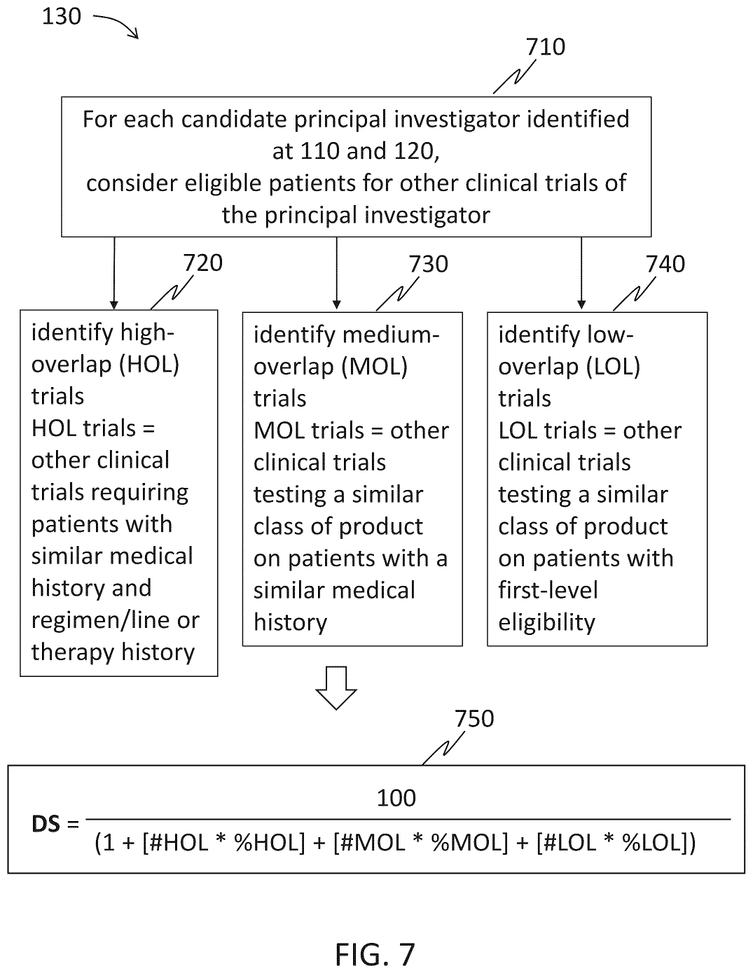

FIG. 7 details aspects of the method 100 related to determining the DS at 130 according to one or more embodiments. At 710, each candidate PI that has been identified (according to the processes at 110 and 120, for example) is considered. More particularly, for each candidate PI, other clinical trials being conducted by the candidate PI are identified and the eligible patients in those other clinical trials are considered. The distraction score (DS) facilitates assessing whether patients of a candidate PI who may otherwise be eligible for the target clinical trial may instead be signed up for another clinical trial. The DS allows determining whether a candidate PI with a given EPS may actually have fewer eligible patients for the target clinical trial due to the distraction of other clinical trials for which those same patients may be eligible.

At 720, identifying high-overlap (HOL) trials refers to identifying other clinical trials of the candidate PI requiring patients with similar medical histories and similar regimen or line or therapy histories. At 730, identifying medium-overlap (MOL) trials refers to identifying other clinical trials of the candidate PI that test a similar class of product on patients with similar medical histories. At 740, identifying low-overlap (LOL) trails refers to identifying other clinical trials of the candidate PI testing a similar class of product on patients with first-level eligibility for the target trial. The identification at 720, 730, 740 and, specifically, the categorization of a trial of the candidate PI as HOL, MOL, or LOL may be based on a trained machine learning model, for example, using features associated with each of the trials of the candidate PI.

At 750, the DS may be computed based on the number of HOL trials (#HOL) and the percentage of overlap (% HOL) assigned to the HOL trials (e.g., 85 percent (#HOL*0.85)), the number of MOL trials (#MOL) and the percentage of overlap (% MOL) assigned to the MOL trials (e.g., 50 percent (#MOL*0.50)), and the number of LOL trials (#LOL) and the percentage of overlap (% LOL) assigned to LOL trials (e.g., 30 percent (#LOL*0.30)). While the example illustrated in FIG. 7 involves three categories into which other clinical trials of each candidate PI may be organized, alternate embodiments may contemplate variations. For example, each of the other clinical trials of the candidate PIs may be assigned a percentage of overlap (e.g., via a machine learning algorithm), and (1*percentage overlap) may be used in the equation for each of the other clinical trials.

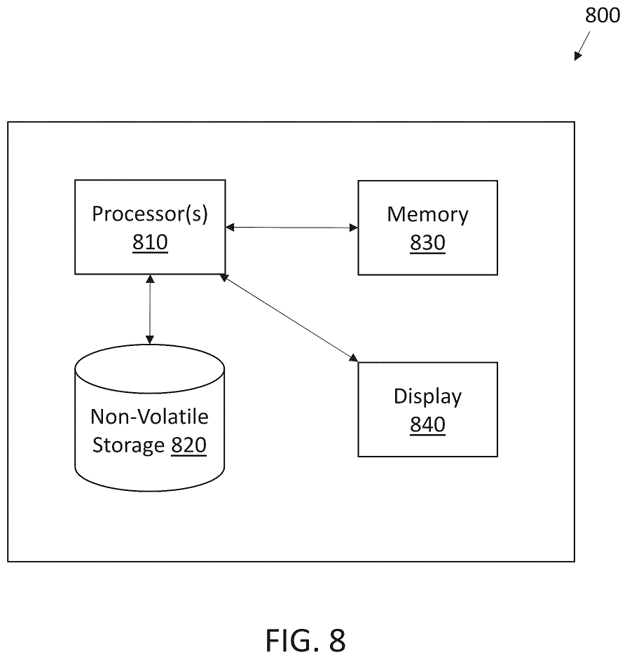

FIG. 8 is a block diagram detailing aspects of a processing system 800 that performs principal investigator identification according to exemplary one or more embodiments. The processing system 800 may include one or more processors 810 that implement the processes shown in FIGS. 1-7, for example. Instructions processed by the one or more processors 810 to implement the method 100 may be stored in non-transitory computer-readable media 820, for example. Any one or more processors 810 may be referred to as “a processor,” and subsequent reference to “the processor” should be interpreted to refer to any one or more of the processors 810. That is different ones of the processors 810 may implement different aspects of the method 100 and other processes discussed herein. Memory 830 may store data and results from implementing the method 100. A display 840 may indicate results of the method 100, for example.

Techniques operating according to the principles described herein may be implemented in any suitable manner. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

Further, some techniques described above comprise acts of storing information (e.g., data and/or instructions) in certain ways for use by these techniques. In some implementations of these techniques—such as implementations where the techniques are implemented as computer-executable instructions—the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

A computing device may comprise at least one processor, a network adapter, and computer-readable storage media. A computing device may be, for example, a desktop or laptop personal computer, a personal digital assistant (PDA), a smart mobile phone, a server, or any other suitable computing device. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

We claim:

1. A computer-implemented method of identifying a principal investigator for a clinical trial, the method comprising:
identifying candidate principal investigators based on similarity of past clinical trials of each of the candidate principal investigators to the clinical trial;
accessing open-source information to identify patients associated with each of the candidate principal investigators;
recording demographic information for the patients associated with each of the candidate principal investigators; and
identifying the principal investigator for the clinical trial based on a match between the demographic information for the patients associated with each of the candidate principal investigators and demographic requirements defined for the clinical trial.

2. The method according to claim 1, wherein the demographic information includes age, gender, race, or ethnicity.

3. The method according to claim 1, further comprising obtaining an overall score for each of the candidate principal investigators based on two or more factors, wherein the overall score includes a weighted sum of the two or more factors.

4. The method according to claim 3, further comprising adjusting weights used in the weighted sum based on the demographic information for the patients associated with each of the candidate principal investigators and demographic requirements defined for the clinical trial.

5. The method according to claim 3, further comprising adjusting the overall score based on the demographic information for the patients associated with each of the candidate principal investigators and demographic requirements defined for the clinical trial.

6. The method according to claim 3, further comprising:
obtaining a first score as one of the two or more factors for each candidate principal investigator among a set of candidate principal investigators, the first score indicating past enrollment success of each candidate principal investigator;
obtaining a second score as one of the two or more factors for each candidate principal investigator, the second score indicating available eligible patients for each candidate principal investigator, wherein each of the eligible patients meets one or more requirements of the clinical study; and
obtaining a third score as one of the two or more factors for each candidate principal investigator, the third score indicating other clinical trials of the candidate principal investigator that involve the eligible patients.

7. The method according to claim 6, wherein obtaining the first score for each candidate principal investigator includes implementing web scraping, filtering, and fuzzy matching using one or more websites that indicate clinical trial details or clinical trial payment information.

8. The method according to claim 6, wherein obtaining the second score for each candidate principal investigator includes:
identifying, among the patients associated with each of the candidate principal investigators, potential eligible patients, according to a diagnosis associated with the potential eligible patients, and eligible patients, as defined by patient requirements of the clinical trial, and
obtaining a weighted sum of a number of the potential eligible patients and the eligible patients associated with the candidate principal investigator.

9. The method according to claim 6, obtaining the third score for each candidate principal investigator includes determining a similarity between the clinical trial and other clinical trials of the candidate principal investigator and computing the third score using a percentage overlap for each of the other clinical trials based on the similarity.

10. The method according to claim 3, further comprising ranking the candidate principal investigators based on the overall score and the demographic information.

11. A non-transitory computer-readable medium storing instructions which, when processed by a processor, cause the processor to implement a method of identifying a principal investigator for a clinical trial, the method comprising:
identifying candidate principal investigators based on similarity of past clinical trials of each of the candidate principal investigators to the clinical trial;
accessing open-source information to identify patients associated with each of the candidate principal investigators;
recording demographic information for the patients associated with each of the candidate principal investigators; and
identifying the principal investigator for the clinical trial based on a match between the demographic information for the patients associated with each of the candidate principal investigators and demographic requirements defined for the clinical trial.

12. The method according to claim 11, wherein the demographic information includes age, gender, race, or ethnicity.

13. The method according to claim 11, further comprising obtaining an overall score for each of the candidate principal investigators based on two or more factors, wherein the overall score includes a weighted sum of the two or more factors.

14. The method according to claim 13, further comprising adjusting weights used in the weighted sum based on the demographic information for the patients associated with each of the candidate principal investigators and demographic requirements defined for the clinical trial.

15. The method according to claim 13, further comprising adjusting the overall score based on the demographic information for the patients associated with each of the candidate principal investigators and demographic requirements defined for the clinical trial.

16. The method according to claim 13, further comprising:
obtaining a first score as one of the two or more factors for each candidate principal investigator among a set of candidate principal investigators, the first score indicating past enrollment success of each candidate principal investigator;
obtaining a second score as one of the two or more factors for each candidate principal investigator, the second score indicating available eligible patients for each candidate principal investigator, wherein each of the eligible patients meets one or more requirements of the clinical study; and
obtaining a third score as one of the two or more factors for each candidate principal investigator, the third score indicating other clinical trials of the candidate principal investigator that involve the eligible patients.

17. The method according to claim 16, wherein obtaining the first score for each candidate principal investigator includes implementing web scraping, filtering, and fuzzy matching using one or more websites that indicate clinical trial details or clinical trial payment information.

18. The method according to claim 16, wherein obtaining the second score for each candidate principal investigator includes:
identifying, among the patients associated with each of the candidate principal investigators, potential eligible patients, according to a diagnosis associated with the potential eligible patients, and eligible patients, as defined by patient requirements of the clinical trial, and
obtaining a weighted sum of a number of the potential eligible patients and the eligible patients associated with the candidate principal investigator.

19. The method according to claim 16, obtaining the third score for each candidate principal investigator includes determining a similarity between the clinical trial and other clinical trials of the candidate principal investigator and computing the third score using a percentage overlap for each of the other clinical trials based on the similarity.

20. The method according to claim 13, further comprising ranking the candidate principal investigators based on the overall score and the demographic information.
